(19) United States
(12) Patent Application Publication
(10) Pub. No.: US 2026/0365547 A1
(43) Pub. Date: Nov. 19, 2026

(54) UV FLUID TREATMENT REACTOR WITH INTERNAL FLANGE FOR MOUNTING A UV RADIATION INSERT

(71) Applicant: TROJAN TECHNOLOGIES GROUP ULC, London (CA)

(72) Inventors: Ayman QURESHI, London (CA); Bojan STOJILJKOVIC, London (CA); Ana-Maria BOGATAN, London (CA); Guang Kuan WEI, London (CA)

(73) Assignee: TROJAN TECHNOLOGIES GROUP ULC, London, ON (CA)

(21) Appl. No.: 19/671,490

(22) Filed: May 8, 2026

Related U.S. Application Data
(60) Provisional application No. 63/805,651, filed on May 14, 2025.

(51) Int. Cl. C02F 1/32; A61L 2/10

(52) U.S. Cl. CPC C02F 1/325; A61L 2/10; A61L 2202/11; A61L 2202/121; A61L 2202/122; A61L 2202/123; C02F 2201/004; C02F 2201/3225; C02F 2303/04

(57) ABSTRACT

An apparatus for treating fluid with ultraviolet (UV) radiation that includes a UV radiation source module that includes a module-side mounting flange and holds at least one UV radiation source that emits UV radiation, a reactor housing that includes an inlet for receiving the fluid, an outlet for discharging treated fluid, and a module opening for receiving the UV radiation source module. The apparatus includes a reactor-side mounting flange that is affixed to the reactor housing and is configured to abut and form a joint with the module-side mounting flange when the UV radiation source module is mounted to the reactor housing, and the reactor-side mounting flange does not extend substantially beyond a perimeter of reactor housing at the module opening.

### CROSS-REFERENCE TO RELATED APPLICATION

This application claims the earlier filing date benefit of U.S. Provisional Application No. 63/805,651, filed on May 14, 2025, the entirety of which is incorporated by reference herein.

### TECHNICAL FIELD

This disclosure relates generally to UV fluid treatment reactors with a mounting structure for the UV lamp insert module that does not have large externally projecting ring flanges, and more particularly relates to UV fluid treatment reactors with an internal flange that creates a joint between the UV reactor housing and the UV lamp insert module.

### BACKGROUND

As the need for cleaner sources of water has increased, water disinfection methods have evolved to match the rising challenge. Water sources may contain heavy metals, sediment, chemicals, pesticides, or the like. Water sources may also contain pathogens such as microorganisms, viruses, or the like. Left untreated, such water may be unhealthy or unsafe for use by humans or animals. Ultraviolet light treatment of water may be used to inactivate pathogens. Water may pass through a small chamber or a larger vessel where the water is subjected to ultraviolet light. The ultraviolet treatment may damage nucleic acids of the pathogens making the pathogens incapable of performing vital cellular functions, thereby rendering them harmless. Thus, this ultraviolet treatment process may make water potable despite the water source containing microorganisms, viruses, or the like.

Existing UV water treatment reactors have a housing that forms a conduit through which the water flows and is exposed to UV light from a UV lamp. In some arrangements, UV lamps may be inserted through a plate that may be mounted onto the housing of the reactor as shown in WO2004/033375A1. As shown in FIG. 4 of this reference, the fluid being treated flows through fluid treatment system in the direction of the arrow B. The fluid treatment system includes a flanged opening 230 with a ring flange 235 that comprises a series of connection openings 237. A UV radiation source module 250 has a flange plate 255 with a series of connection openings 257. The UV radiation source module 250 can be inserted into the flanged opening 230 so that the ring flange 235 on the reactor abuts the flange plate 255 of the radiation source module 250. These two flange plates can form a joint that mounts the radiation source module 250, e.g., by connecting the two flange plates 235, 255 with bolts through their connection openings 257 and 237. The flange plate 255 comprises openings through which lamps and protective sleeves may be inserted into the reactor without removing flange plate 255.This is a typical arrangement in available UV water treatment reactors where the UV lamp insert includes a blind flange (flat plate) that mates to a ring flange that is typically separately welded or jointed to the exterior of the reactor.

WO2004/033375A1 shows an example of a cross-flow reactor, but similar external flange mounting structures are also used in parallel flow or counter flow reactors. Regardless of the type of flow, external flanges are used to mount the source module on the side of the reactor, in the middle of the reactor (e.g., as in EP2368852), or at an end of the reactor. For pressure-rated treatment systems the flanges must meet certain standards such as ASTM, ASME, or ANSI. These standards set minimum thickness values that increase with the design pressure of the system and the diameter of the flange. In particular, the flat (blind) flange plate 255 must withstand the pressure without significant deflection. As a result, these blind flange plates must be very thick, heavy, and expensive. The weight of a large flange may require an overhead crane for removal and installation. It would be beneficial to reduce the cost and weight of these flanges while still meeting the requirements for pressure rating standards.

This issue can also be seen from the reactor described in WO1996011879A1. FIG. 1 of this reference shows that a flange plate 28 is fastened to an external ring flange 26 using bolts 30. This figure shows that the ring flange 26 and the flange plate 28 are larger in diameter than the reactor housing 12.

### SUMMARY

The flanges that are used to mount the UV radiation module are heavy, large, and expensive, and two flanges are required for each joint. The inventors discovered an improved way of affixing the UV radiation module to the reactor without using external ring flanges.

In one aspect, this disclosure provides an apparatus for treating fluid with ultraviolet (UV) radiation, which includes (i) a UV radiation source module that includes a module-side mounting flange and is configured to hold at least one UV radiation source that emits UV radiation; (ii) a reactor housing comprising an inlet for receiving the fluid, an outlet for discharging treated fluid, and a module opening for receiving the UV radiation source module so that the UV radiation source module can be mounted to the reactor housing; and (iii) a reactor-side mounting flange that is affixed to the reactor housing and is configured to abut and form a joint with the module-side mounting flange when the UV radiation source module is mounted to the reactor housing. The reactor-side mounting flange does not extend substantially beyond a perimeter of the reactor housing at the module opening.

### BRIEF DESCRIPTION OF THE DRAWINGS

FIG. 1 is a schematic diagram of a conventional apparatus for treating fluid with UV radiation;

FIG. 2 is a schematic diagram of an apparatus for treating fluid with UV radiation according to one embodiment of the invention;

FIG. 3 is a perspective of the apparatus for treating fluid with UV radiation without the UV radiation source module;

FIG. 4 is a front view of a UV radiation source module according to another embodiment; and

FIG. 5 is a schematic diagram of an apparatus for treating fluid with UV radiation according to another embodiment of the invention,

### DETAILED DESCRIPTION OF EMBODIMENTS

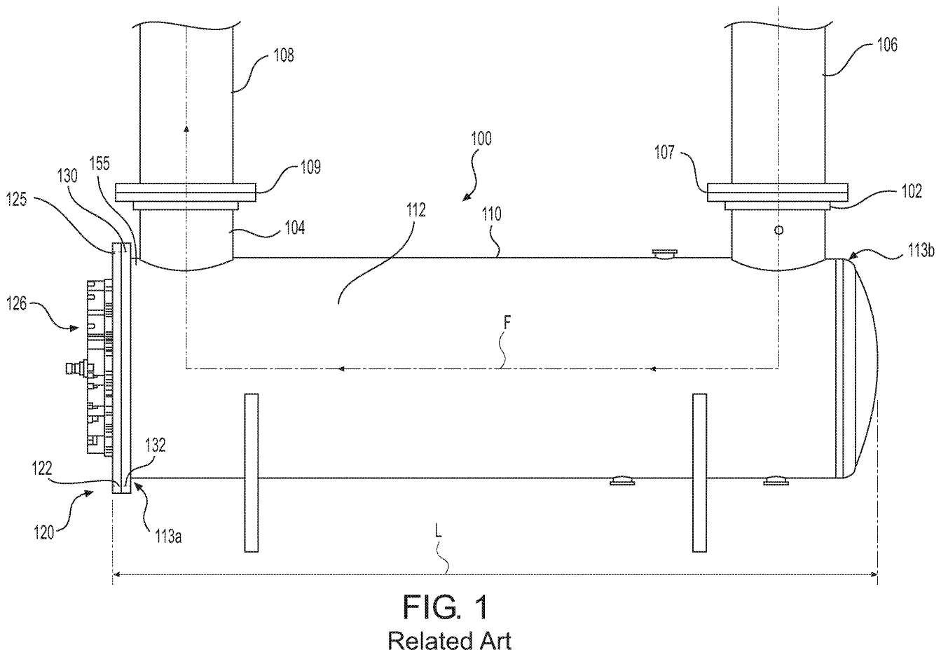

For purposes of illustration, FIG. 1 shows a conventional apparatus for treating fluid with ultraviolet (UV) radiation 100 that includes a reactor housing 110 and a UV radiation source module 120. The housing 110 includes an inlet 102 for receiving a fluid to be treated (e.g., water) from conduit 106. The conduit 106 can form joint 107 with the inlet 102. The fluid, F, flows within the reactor housing 110 and exits the reactor housing 110 at outlet 104. The outlet 104 can discharge the treated fluid to outlet conduit 108, which can form a joint 109 with the outlet 104.

As can be seen in FIG. 1, the apparatus 100 is an elongate tubular structure defined by a reactor housing 110 that extends in a lengthwise dimension L and has a tubular shaped side wall 112, a first end 113a in the lengthwise direction, and a second end 113b in the lengthwise direction. A module opening (not shown in FIG. 1) is formed in the first end 113a of the reactor housing 110. In FIG. 1, the UV radiation source module 120 covers the module opening. An internal reactor-side flange 130 has been welded or otherwise permanently affixed to the first end 113a of the reactor housing and includes a ring that has a width extending radially outwardly with respect to the perimeter 155 of the reactor housing 100 at the module opening. The reactor-side flange 130 includes a plurality of openings (bolt holes) 132 extending around the circumference of the flange for receiving fasteners. The UV radiation source module 120 includes a flat flange plate 125 (blind flange) with a plurality of openings 122 for receiving fasteners, which are aligned with the plurality of openings 132 on the reactor-side flange 130. The openings 132 in the reactor-side flange 130 can be threaded to receive threaded bolts as fasteners, for example, to mount the UV radiation source module 120 to the reactor housing 110. Alternatively, the holes may not be threaded, in which case bolts with matching nuts are used to secure flange plate 125 to plate 130. As can be seen, the flange plate 125 and the reactor-side flange 130 have larger diameters than the reactor housing 110.

The UV radiation source module 120 also includes a plurality of holes 126 with sleeves or bushings for inserting and holding UV lamps or sleeves that extend into the reactor housing 110 and emit UV light to treat the fluid.

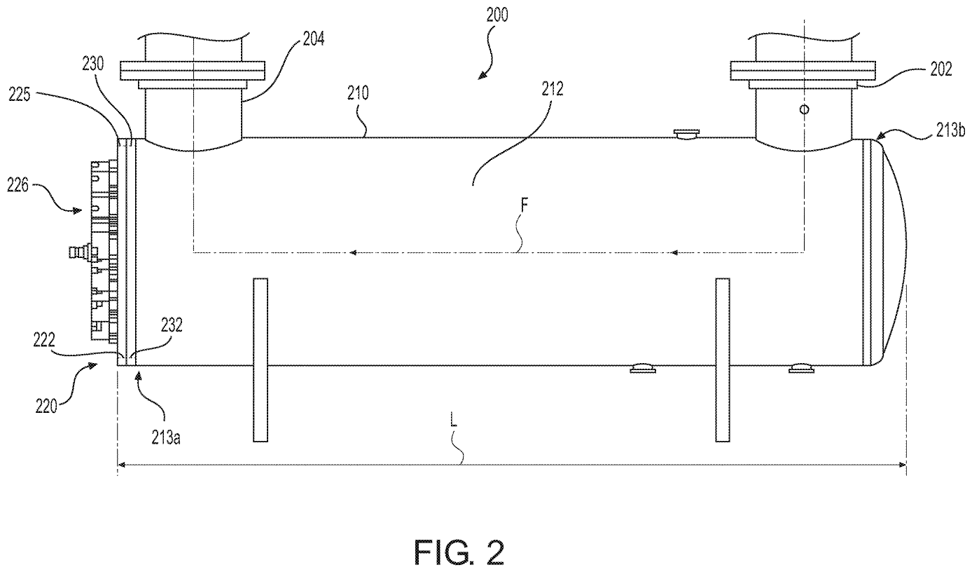

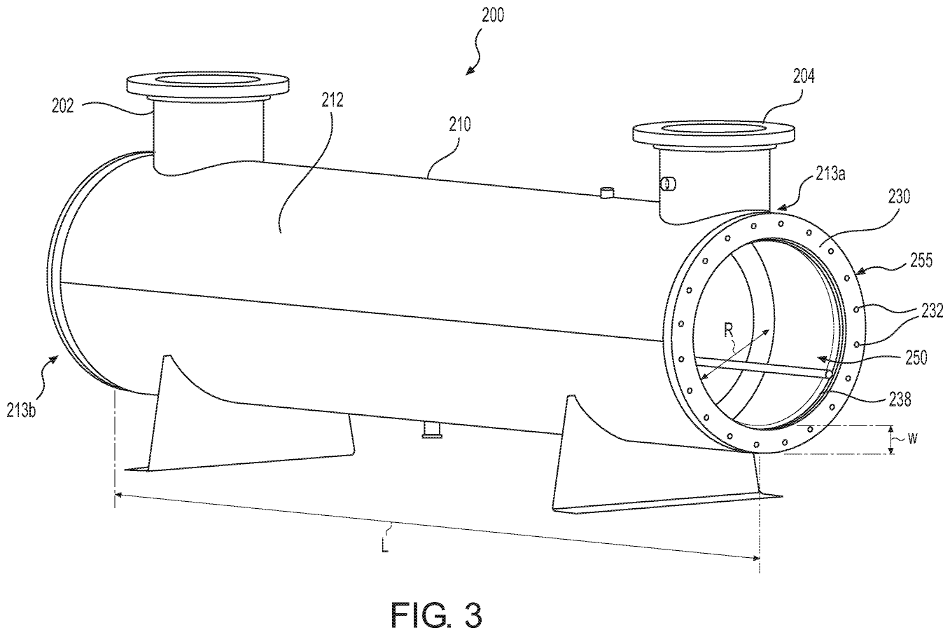

FIGS. 2 and 3 illustrate an apparatus 200 for treating fluid with UV light according to one embodiment of the invention. FIG. 2 is a schematic side view of the apparatus 200 and FIG. 3 is a perspective view of the apparatus 200 without the UV radiation source module 220. The apparatus 200 includes a reactor housing 210 and a UV radiation source module 220. The housing 210 includes an inlet 202 for receiving a fluid to be treated (e.g., water) from a conduit. The fluid, F, flows within the reactor housing 210 and exits the reactor housing 210 at outlet 204 after being treated with UV radiation.

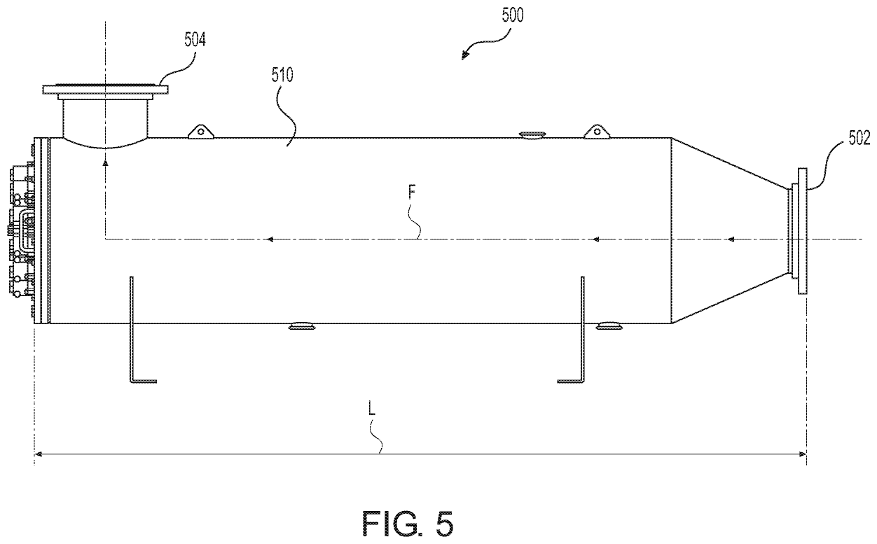

The reactor housing 210 extends in a lengthwise dimension L and has an elongate tubular shaped side wall 212, a first end 213a in the lengthwise direction, and a second end 213b in the lengthwise direction. As shown, the inlet 202 is oriented radially relative to the lengthwise direction L, but could also be oriented in the lengthwise direction L as shown in FIG. 5. A module opening 250 (FIG. 3) is formed in the first end 213a of the reactor housing 110. In FIG. 2, the UV radiation source module 220 covers the module opening 250. An internal reactor-side flange 230 has been welded or otherwise permanently affixed to the first end 213a of the reactor housing. In other embodiments, the reactor-side flange 230 could be arranged and affixed to the reactor housing 110 so that it is recessed from the end of the reactor somewhat.

The UV radiation source module 220 includes a flat flange plate 225 (blind flange) with a plurality of openings 222 for receiving fasteners, which are aligned with a plurality of openings 232 on the reactor-side flange 230. The openings 232 in the reactor-side flange 230 can be threaded to receive threaded bolts as fasteners, for example, to mount the UV radiation source module 220 to the reactor housing 210. Alternatively, as described in FIG. 1, the openings 232 do not necessarily need to be threaded and nuts can be used to secure the flange plate 225 to the reactor-side flange 230. The plurality of openings 222, 232 are positioned radially inwardly of the reactor housing perimeter 255. When the UV radiation source module 220 is mounted to the reactor housing 210, the inwardly facing side of the flange plate 225 abuts the outwardly facing side of the reactor-side flange 230, and this joint can form a liquid-tight seal between the UV radiation source module 220 and the reactor housing 210.

The UV radiation source module 220 includes a plurality of holes 226 with sleeves or bushings for inserting and holding UV lamps or sleeves that extend into the reactor housing 210 and emit UV light to treat the fluid.

The outer diameter of the reactor-side flange 230 is substantially the same as the outer diameter of the reactor housing 210 at the module opening 250. In embodiments, the width, w, of the reactor-side flange that extends in the radial direction R can be in a range of from 1% to 20% of the diameter of the reactor housing perimeter 255 or from 5% to 15% of the diameter of the reactor housing perimeter 255. The inner edge 238 (inner perimeter) of the reactor-side flange 230 extends radially inwardly of the reactor housing 210.

As can be seen in FIGS. 2 and 3, in contrast to conventional UV reactors (FIG. 1), the reactor-side flange 230 and the flange plate 225 do not extend radially beyond the perimeter 255 of the reactor housing 210 at the module opening 250, and instead are substantially flush with the perimeter 255 of the reactor housing 210 at the module opening 250. In this case, an outer diameter of the reactor-side flange 230 and the diameter of flange plate 225 are the same as the diameter of the reactor housing.

In other embodiments, the flanges do not need to be flush with the reactor housing and could alternatively extend inwardly of the reactor housing perimeter (i.e., the outer diameter of the reactor-side flange and/or the diameter of the flange plate could be less than the diameter of the reactor housing at the module opening). Also, in some embodiments the reactor-side flange can be dimensioned and affixed to the reactor housing so that it extends slightly, but not substantially outwardly from the module opening beyond the perimeter of the reactor housing, i.e., “substantially” in this context means that a dimension of any portion of the flange that extends beyond the reactor housing should be less than 5% of the longest dimension of the module opening. In embodiments, the flange on the UV radiation module should also not substantially extend (i.e., less than 5%) outwardly from the module opening beyond the perimeter of the reactor housing. Thus, in contrast with the prior art discussed above, since outwardly projecting flanges are not used, the reactor-side flange and the flange plate of the UV radiation source module can be made to be smaller and lighter, which also saves costs. Smaller diameter flanges can be thinner while achieving pressure ratings, which further reduces their weight and cost.

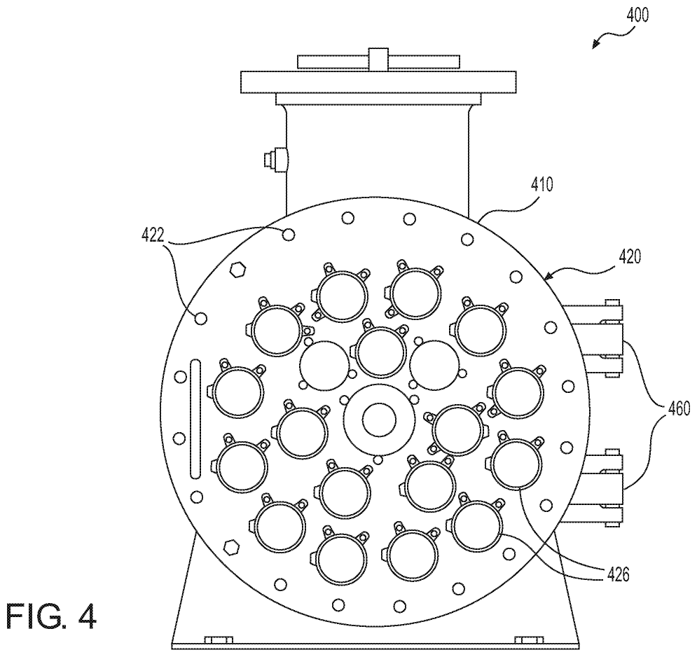

FIG. 4 shows a front view of UV radiation source module 420 of apparatus 400 according to another embodiment. As in the prior embodiment, the UV radiation source module 420 includes a plurality of fastener openings 422 and a plurality of openings 426 for holding and inserting UV radiation sources (e.g., rod-shaped UV lamps). In this embodiment, the UV radiation source module 420 is also mounted to the reactor housing 410 by hinges 460. The hinges 460 can be attached directly to the reactor housing 410 or can be a part of the reactor-side flange that is affixed to the reactor housing 410. This eliminates the need for an overhead crane to lift and hold the flange plate when it is removed for service.

FIG. 5 shows a side view of an apparatus 500 for treating fluid with UV light according to another embodiment. The apparatus 500 in FIG. 5 has the same features as the embodiment illustrated in FIGS. 2 and 3 except that inlet 502 is oriented in the lengthwise direction L of the reactor housing 510. As schematically illustrated, the fluid, F, flows in the inlet 501 of the apparatus 500, through the reactor housing 510, and exits the reactor housing 510 at outlet 504 after being treated with UV radiation.

In the embodiments shown in FIGS. 1-5, the module opening is formed in an end of the reactor, the UV radiation module is mounted to the reactor housing in an axial or lengthwise direction of the reactor housing and the UV lamps are elongate rods that extend primarily in a lengthwise in a direction of the reactor. In other embodiments, the module opening can be formed on the side of the reactor housing, so that the UV radiation module is mounted in a direction perpendicular to the lengthwise direction and the UV lamps can extend primarily in a direction perpendicular to the major flow direction. Additionally, in other embodiments, the apparatus can have a plurality of module openings to receive a plurality of UV radiation source modules, and the UV lamps can be any shape, such as rod-shaped, puck-shaped, etc.

It will be appreciated that the above-disclosed features and functions, or alternatives thereof, may be desirably combined into different apparatuses. Also, various alternatives, modifications, variations or improvements may be subsequently made by those skilled in the art, and are also intended to be encompassed by the disclosed embodiments. As such, various changes may be made without departing from the spirit and scope of this disclosure.

What is claimed is

1. An apparatus for treating fluid with ultraviolet (UV) radiation, the apparatus comprising:
a UV radiation source module that includes a module-side mounting flange and is configured to hold at least one UV radiation source that emits UV radiation;
a reactor housing comprising an inlet for receiving the fluid, an outlet for discharging treated fluid, and a module opening for receiving the UV radiation source module so that the UV radiation source module can be mounted to the reactor housing; and
a reactor-side mounting flange that is affixed to the reactor housing and is configured to abut and form a joint with the module-side mounting flange when the UV radiation source module is mounted to the reactor housing;
wherein the reactor-side mounting flange does not extend substantially beyond a perimeter of the reactor housing at the module opening.

2. The apparatus of claim 1, wherein the module-side mounting flange does not extend substantially beyond a perimeter of the reactor housing at the module opening when the UV radiation source module is mounted to the reactor housing.

3. The apparatus of claim 1, wherein the module-side mounting flange includes a plurality of openings for receiving fasteners that are able to fasten the module-side mounting flange to the reactor-side mounting flange.

4. The apparatus of claim 3, wherein the reactor-side mounting flange includes a plurality of threaded openings that align with the plurality of openings on the module-side mounting flange when the UV radiation source module is removably mounted to the reactor housing.

5. The apparatus of claim 4, wherein the plurality of threaded openings are positioned inwardly from the perimeter of the reactor housing at the module opening.

6. The apparatus of claim 1, wherein the module-side mounting flange is a flange plate having at least one opening for receiving the at least one UV radiation source such that the UV radiation source is immersed in the fluid while it is being treated.

7. The apparatus of claim 6, wherein the flange plate forms a seal with reactor housing when the UV radiation source module is mounted to the reactor housing.

8. The apparatus of claim 1, wherein the reactor housing forms an elongate conduit through which the fluid flows from the inlet to the outlet, and wherein the module opening is located on an end of the reactor housing in a lengthwise direction.

9. The apparatus of claim 1, wherein the reactor housing forms an elongate conduit through which the fluid flows from the inlet to the outlet, and wherein the module opening is located on a side of the reactor housing so that the UV radiation source module is mounted to the reactor housing in a direction that is perpendicular to a lengthwise direction.

10. The apparatus of claim 1, wherein the reactor-side mounting flange has an outer edge that is flush with a perimeter of reactor housing at the module opening.

11. The apparatus of claim 1, wherein the module-side mounting flange has an outer edge that is flush with a perimeter of reactor housing at the module opening.

12. The apparatus of claim 1, wherein the UV radiation source module is mounted to at least one of the reactor housing and reactor-side mounting flange by one or more hinges.
